(19) United States
(12) Patent Application Publication
(10) Pub. No.: US 2013/0150698 A1
(43) Pub. Date: Jun. 13, 2013

(54) EVENT-BASED BIO-SIGNAL CAPTURING SYSTEM

(71) Applicant: OP GLOBAL HOLDINGS LIMITED, Road Town (VG)

(72) Inventors: Fu-Chieh Hsu, Stateline, NV (US); Shauh-Teh Juang, Saratoga, CA (US); Mark Shane Peng, Hsinchu County (TW)

(73) Assignee: OP GLOBAL HOLDINGS LIMITED, Road Town (VG)

(21) Appl. No.: 13/674,242

(22) Filed: Nov. 12, 2012

Related U.S. Application Data
(60) Provisional application No. 61/568,255, filed on Dec. 8, 2011.

(51) Int. Cl. A61B 5/11; G06F 19/00; A61B 5/0408; A61B 5/0478; A61B 5/04; A61B 5/0492

(52) U.S. Cl. CPC A61B 5/11; A61B 5/04; A61B 5/0492; A61B 5/0408; A61B 5/0478; A61B 5/1116; G06F 19/00
USPC 600/390; 600/393; 600/391; 600/595; 702/19

(57) ABSTRACT

An event-based bio-signal capturing system is disclosed. At least one bio-signal capturing device captures a bio-signal measured from biological beings; and at least one event capturing device captures an event and generates a corresponding event marker. A data recording device acquires the bio-signal and the event marker, wherein the bio-signal and the event marker are acquired with corresponding time reference for subsequent event-based data analysis.

### BACKGROUND OF THE INVENTION

1. Field of the Invention

The present invention generally relates to a bio-signal capturing system, and more particularly to an event-based bio-signal capturing system.

2. Description of Related Art

Current bio-signal applications for human beings are mainly focusing in the following areas: (1) bulky systems for formal medical practices, (2) rudimentary gadgets for fitness monitoring, and (3) bulky brainwave-based gadgets for rudimentary applications of brainwave signal. These devices and applications are based largely on bio-signal only. There is no user-friendly and wearable system or product on the market that correlates the bio-signal with other parameters to obtain accurate identification on stimuli of bio-signal for applications require robust and consistent interpretation of bio-signal.

For the foregoing reasons, a need has arisen to propose a novel bio-logical capturing system, for example, to achieve more compact, wearable and suitable for mobile uses like fitness monitoring, sports monitoring and game playing, and also provides a comfortable setting for stationary uses like sleep monitoring and meditation monitoring.

### SUMMARY OF THE INVENTION

In view of the foregoing, it is an object of the embodiment of the present invention to provide an event-based bio-signal capturing system that leverages both bio-signal of biological beings and event marker from the user or the environment. The embodiment provides accurate identification on stimuli of bio-signal for applications require robust and consistent interpretation of bio-signal.

According to one embodiment, an event-based bio-signal capturing system includes at least one bio-signal capturing device, at least one event capturing device, and a data recording device. The bio-signal capturing device is configured to capture a bio-signal measured from biological beings. The event capturing device is configured to capture an event and generate a corresponding event marker. The data recording device is configured to acquire the bio-signal and the event marker. The bio-signal and the event marker are acquired with corresponding time reference for subsequent event-based data analysis.

### BRIEF DESCRIPTION OF THE DRAWINGS

FIG. 1 shows a block diagram illustrating an event-based bio-signal capturing system according to one embodiment of the present invention;

FIG. 2 shows exemplary acquisition with time reference;

FIG. 3 shows an exemplary event-based bio-signal capturing system;

FIG. 4A and FIG. 4B show a top view and a side view of the bio-signal capturing device, respectively, according to one embodiment of the present invention;

FIG. 5A and FIG. 5B show wearable bio-signal capturing devices; and

FIG. 6A and FIG. 6B show a top view and a side view of the bio-signal capturing device, respectively, according to another embodiment of the present invention.

### DETAILED DESCRIPTION OF THE INVENTION

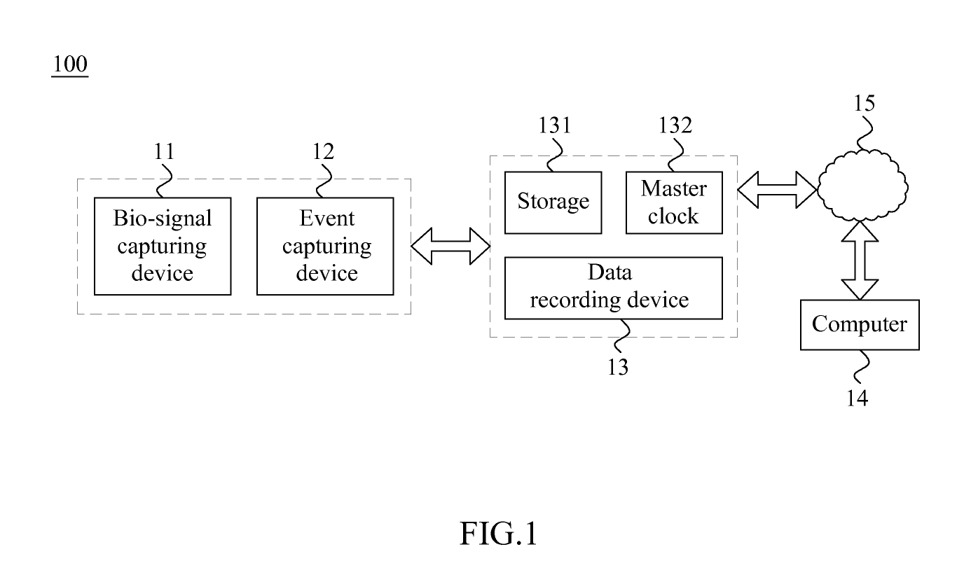

FIG. 1 shows a block diagram illustrating an event-based bio-signal capturing system 100 according to one embodiment of the present invention. In the embodiment, the event-based bio-signal capturing system (or “system” hereinafter) 100 includes at least one bio-signal capturing device 11 and at least one event capturing device 12. Specifically, the bio-signal capturing device 11 is configured to capture a bio-signal measured from biological beings such as human beings; and the event capturing device 12 is configured to capture an event and generate a corresponding event marker that indicates a specific time and/or place of the associated bio-signal. Although one bio-signal capturing device 11 and one event capturing device 12 are shown in figure, it is appreciated that the amount of the bio-signal capturing device 11 and the amount of the event capturing device 12, respectively, may be greater than one, and their amounts may be different from each other. The bio-signal capturing device 11 and the event capturing device 12 may be separated from each other, but operated locally, for example, within personal reach. Alternatively, the bio-signal capturing device 11 and the event capturing device 12 may be integrated into a single module.

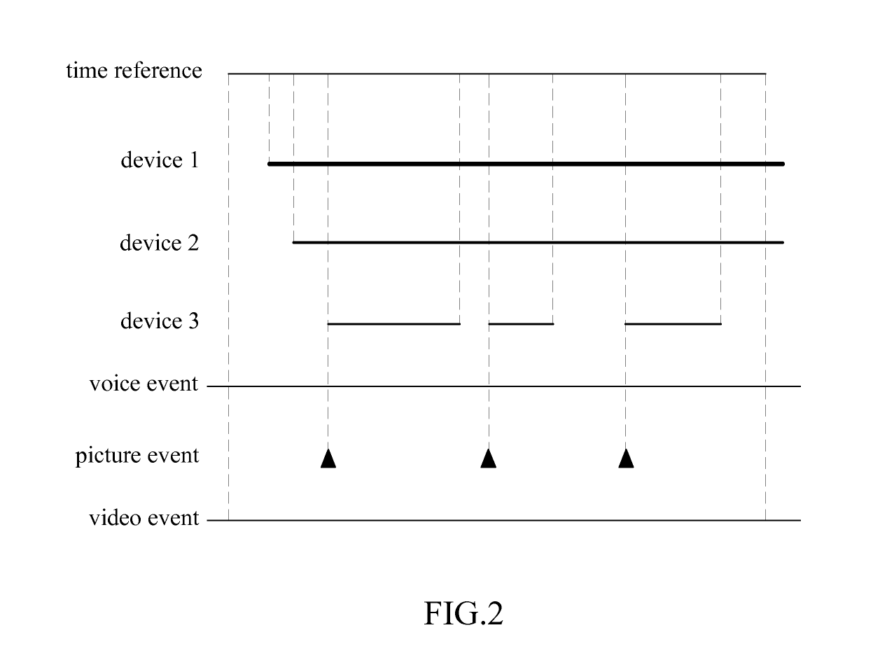

The system 100 may further include a data recording device 13 that is configured to acquire the captured bio-signal and event marker from the bio-signal capturing device 11 and the event capturing device 12, respectively. The acquired bio-signal and the event marker may be further stored, with or without data compression, for example, in a storage area 131 associated with the data recording device 13. Moreover, the bio-signal and the event marker are acquired with corresponding time reference for subsequent event-based data analysis. The time reference may, for example, derived from a master clock 132 associated with the data recording device 13. FIG. 2 shows exemplary acquisition with time reference that synchronizes or aligns bio-signals from three bio-signal capturing devices 11 (e.g., a device 1, a device 2 and a device 3) and three event markers (e.g., a voice event, a picture event and a video event). The acquisition of the bio-signal and event marker by the data recording device 13 may be accomplished in a wired manner, a wireless manner or their combination.

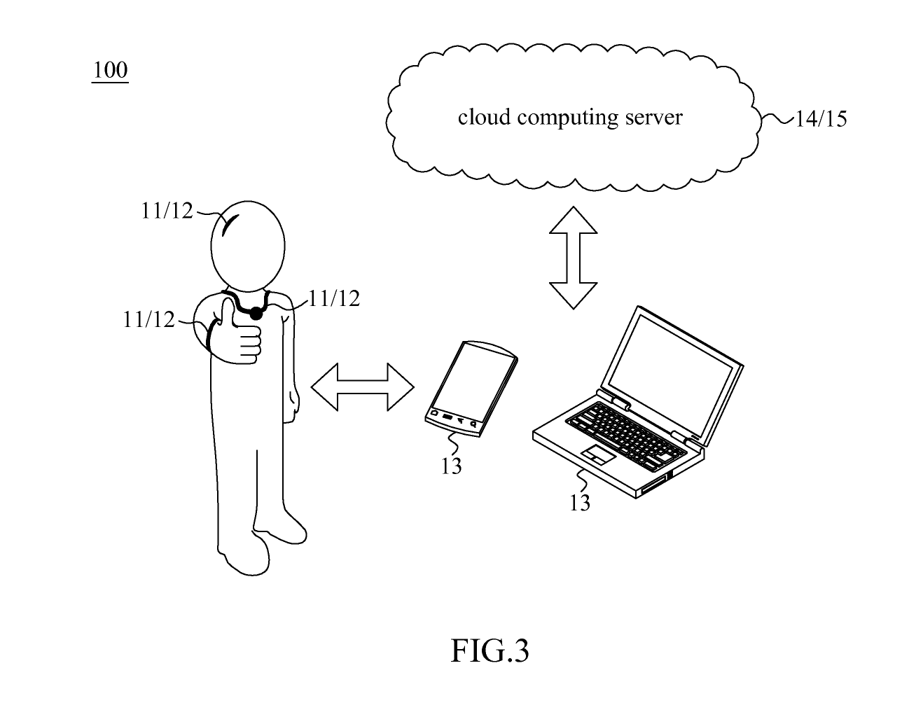

The acquired bio-signal and the event marker may be subjected to data analysis in the data recording device 13, such as a portable computing device (e.g., a mobile phone) for mobile applications or a personal computer for stationary applications. Alternatively, the data analysis may be performed by a computer 14 that is communicated with the data recording device 13 via a computer network 15 such as the Internet or an intranet. FIG. 3 shows an exemplary system 100, in which the bio-signals and the event markers are provided from integrated bio-signal capturing device and event capturing device 11/12 to the data recording device 13, and are further subjected to data analysis in cloud computing servers 14/15. In the shown example, the integrated bio-signal capturing device and event capturing device 11/12 are, for example, wearable and mobile devices.

The bio-signal capturing device 11 mentioned above may capture electrical-activity bio-signals such as Electroencephalography (EEG) signals, Electrocardiogram (ECG) signals or Electromyography (EMG) signals. Alternatively or combinedly, the bio-signal capturing device 11 may capture physiology bio-signals such as skin electrical potential signals, skin conductance (SC) signals, blood flow signals, oxygen content signals or body temperature signals.

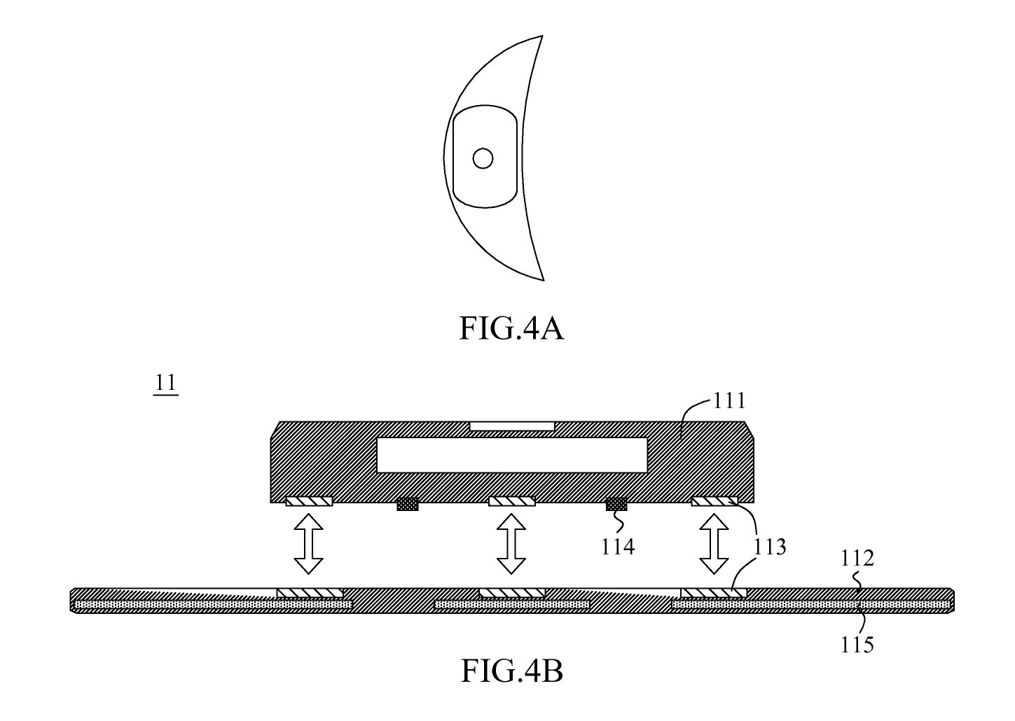

FIG. 4A and FIG. 4B show a top view and a side view of the bio-signal capturing device 11, respectively, according to one embodiment of the present invention. In the embodiment, the bio-signal capturing device 11 includes an electronic module 111 and a sensing patch 112. The electronic module 111 may be attached to or detached from the sensing patch 112 through electrodes (or internal electrodes) 113, such as magnetized electrodes, for maintenance of the electronic module 111, replacing the existing sensing patch 112 or switching to different type of sensing patch 112. The electronic module 111 may further include charging electrodes 114 for power charging a rechargeable battery (not shown). Generally speaking, the bio-signal capturing device 11 may be powered by battery, external power, wireless energy source, or energy harvesting mechanism. The sensing patch 112 may include conductive means 115 (e.g., conductive adhesive gel), which is electrically coupled with the electrode 113, for making electrical contacts to skin, for applications requiring secure contact at fixed body location.

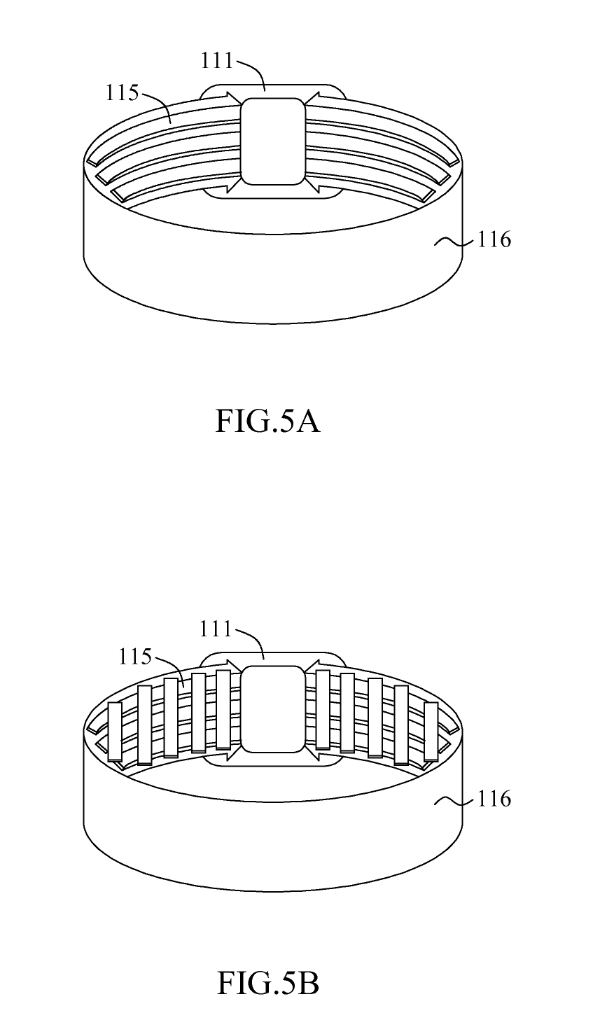

As shown in FIG. 5A or FIG. 5B, the bio-signal capturing device 11, configured as a wearable device, may further include holding means 116 for holding the conductive means 115 firmly to skin. The holding means 116, for example, may be medical adhesive tape, elastic band or elastic string. For applications requiring loose contact at approximate body location, the sensing patch 112 is equipped with plural electrodes (or external electrodes) 115 that have configurations of bands, patches, dots, meshes or their combination in order to maximize extent of contact.

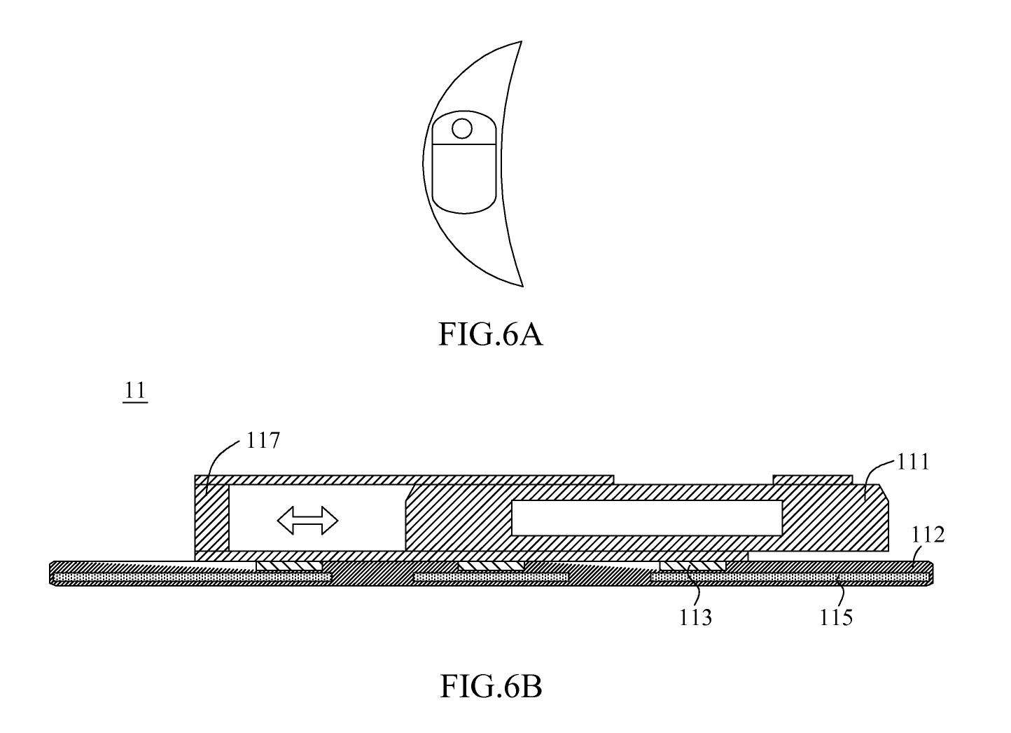

FIG. 6A and FIG. 6B show a top view and a side view of the bio-signal capturing device 11, respectively, according to another embodiment of the present invention. The present embodiment is similar to the preceding embodiment (FIG. 4A/B) with the following distinction. In the present embodiment, the electronic module 111 may be attached to or detached from the sensing patch 112 through a socket connector 117 or individual conducting miniature sockets (not shown).

As mentioned above, there may have a variety of possible bio-signals captured from the bio-signal capturing device 11. Accordingly, the sensing patch 112 may be designed or manufactured as an EEG patch, an ECG patch with attenuator or reduced gain, an EMG patch with attenuator or reduced gain, a skin-conductance patch with active signal source (clock) or power source, a blood flow patch with light emitting diodes (LED) and transducer, an oxygen content patch with LED and transducer, or a general transducer patch with micro controller unit (MCU) and transducers.

The event capturing device 12 mentioned above may capture one or more events in the following (nonexclusive) list: voice recording, sound recording, still imagery, video recording, body/muscle movement or posture, electromagnetic field (EMF) exposure, geographic location, orientation/gesture, finger tapping, altitude, temperature, humidity, and air pressure. The event capturing device 12 may include a rechargeable battery (not shown), and, generally speaking, may be powered by battery, external power, wireless energy source, or energy harvesting mechanism.

The event capturing device 12 mentioned above may be running continuously, or be activated by one or more ways in the following (nonexclusive) list: user, voice, sound, scene change, body/muscle movement or posture, EMF exposure change, geographic location change, orientation change, altitude change, temperature change, humidity change, air pressure change, computer software, and preset conditions.

With respect to the event-based data analysis mentioned above, in one exemplary embodiment, the data recording device 13 performs a bio-signal/event correlation analysis based on the time reference of the captured bio-signal and event marker. In another exemplary embodiment, the data recording device 13 performs multi-dimensional pattern recognition based on a priori characterization and modeling of the captured bio-signal and event marker. A variety of data analyses may be adapted to the event-based data analysis such as sleep analysis, meditation analysis, mood analysis, stress and relaxation analysis, bio-feedback, fitness analysis, attention analysis or interactive game playing.

In order to make the event-based bio-signal capturing system 100 more compact and mobile, some energy saving techniques are deployed in the following exemplary embodiments. The bio-signal capturing device 11 and the event capturing device 12 commonly utilize an analog-to-digital converter (ADC) for converting input continuous physical quantity to a digital number that represents the quantity's amplitude.

In one exemplary embodiment, at least one of the bio-signal capturing device 11 and the event capturing device 12 adopts dynamic gain switching during ADC sampling. Specifically speaking, a high resolution ADC may consume much higher power than a medium resolution ADC. For example, a 16-bit resolution ADC may consume up to 64 times more power than a 10-bit resolution ADC. Since many bio-signals combine occasional large swings (that carry less information content) and mostly smaller swings (that carry more information content), a sampling algorithm using dynamic gain switching (10X for small swing signals and 1X resampling for large swing signals if the 10X gain causes out-of-range condition) using 10-bit resolution ADC can achieve effective 13.5-bit dynamic range that uses only slightly more than the power consumption of 10-bit resolution ADC.

In another exemplary embodiment, at least one of the bio-signal capturing device 11 and the event capturing device 12 adopts non-uniform quantization for ADC sampling. Specifically speaking, by using an algorithm with gain cross-over hysteresis (to reduce the probability of double sampling), the occasional double-sampling power increase is less than 5% of that of a 10-bit ADC, and a power savings of up to 64 times than that when a 16-bit ADC is used.

In a further exemplary embodiment, at least one of the bio-signal capturing device 11 and the event capturing device 12 adopts data scrambling to prevent consecutive sampling data points being closest neighbors during communication; and the data recording device 13 adopts spike removal within a corrupted data packet to reduce noise energy. Specifically speaking, for communications in a noisy environment, there may be frequent interferences from many RF spike/bursting sources (such as WiFi, cell-phone, Blue-Tooth, WiMAX devices) as well as other communication noise sources. Traditional communication techniques rely on Cyclic Redundancy Check (CRC) or other error-detection coding and detection methods to detect whether a data packet is corrupted. The corrupted data packets will either be lost or require energy-consuming communication handshake and re-transmissions. Since bio-signal sampling rate is generally much higher than the frequency contents of bio-signals to minimize sampling noise and Nyquist aliasing, the present exemplary embodiment thus implements algorithm with data scrambling within data packet (that is, the captured bio-signal sampled data points are rearranged within data packet, so that no two consecutive sampled data points are nearest neighbors to each other, and thus resulting in very few consecutive sampled data point corruptions), plus spike removal algorithm to repair corrupted data packet (that is, to detect which sampled data point is corrupted and to repair it with minimum noise energy). This algorithm enables high-quality signal reception in a noisy environment.

In a further exemplary embodiment, at least one of the bio-signal capturing device 11 and the event capturing device 12 adopts collision detection, and adopts skip-forward algorithm upon collision detection. Specifically speaking, in order to conserve energy consumption and to minimize interferences from multiple bio-signal and event capturing devices 11/12, the present exemplary embodiment thus implements collision detection and time hopping communication algorithm. To conserve energy, each capturing device 11/12 will only turn on momentarily (less than 10% for ADCs) to capture the intended signal or event, while staying in low power mode most of the time. Similarly, the communication or storage circuits will only turn on momentarily (less than 1% for communication circuit and less than 5% for storage circuit) to transmit or store the captured signals and events, while staying in low power mode most of the time. Because all capturing devices 11/12 operate with their own fixed routines independently (that is, asynchronously), and with their time bases not perfectly matched (some faster and some slower), all these capturing devices 11/12 will gradually run into each other (systematically) causing collisions in communications and possible data packet corruptions. In the present embodiment, each capturing device 11/12, prior to start of communication transmission phase, will first check if there is any other device transmitting (possible collision) without receiving any data (that is, just to detect carrier signal that may corrupt its transmission). As any device 11/12 detecting such carrier is most likely the faster device (for it's chasing up from behind), it will skip one or more sampling data points (therefore “hasten up” or skip-forward) for its next data packet transmission without causing any actual collision nor losing any data packet. For devices with <1% communication circuit duty cycle, this algorithm allows more than 50 devices per data channel operating simultaneously (and asynchronously) without having collision or data corruptions.

Although specific embodiments have been illustrated and described, it will be appreciated by those skilled in the art that various modifications may be made without departing from the scope of the present invention, which is intended to be limited solely by the appended claims.

What is claimed is:

1. An event-based bio-signal capturing system, comprising:
at least one bio-signal capturing device configured to capture a bio-signal measured from biological beings;
at least one event capturing device configured to capture an event and generate a corresponding event marker; and
a data recording device configured to acquire the bio-signal and the event marker;
wherein the bio-signal and the event marker are acquired with corresponding time reference for subsequent event-based data analysis.

2. The system of claim 1, wherein the event marker indicates a specific time and/or place of the associated bio-signal.

3. The system of claim 1, wherein the bio-signal capturing device and the event capturing device are separated from each other, and are operated locally.

4. The system of claim 1, wherein the bio-signal capturing device and the event capturing device are integrated into a single module.

5. The system of claim 1, further comprising a storage area associated with the data recording device for storing the bio-signal and the event marker.

6. The system of claim 1, further comprising a master clock associated with the data recording device for deriving the time reference.

7. The system of claim 1, wherein the bio-signal and the event marker are acquired by the data recording device in a wired manner.

8. The system of claim 1, wherein the bio-signal and the event marker are acquired by the data recording device in a wireless manner.

9. The system of claim 1, wherein the event-based data analysis is performed in the data recording device.

10. The system of claim 1, further comprising:
a computer configured to perform the event-based data analysis; and
a computer network, via which the computer is communicated with the data recording device.

11. The system of claim 1, wherein the bio-signal is an electrical-activity bio-signal.

12. The system of claim 11, wherein the electrical-activity bio-signal is an Electroencephalography (EEG) signal, an Electrocardiogram (ECG) signal or an Electromyography (EMG) signal.

13. The system of claim 1, wherein the bio-signal is a physiology bio-signal.

14. The system of claim 13, wherein the physiology bio-signal is a skin electrical potential signal, a skin conductance (SC) signal, a blood flow signal, an oxygen content signal or a body temperature signal.

15. The system of claim 1, wherein the bio-signal capturing device comprises:
a sensing patch;
an electronic module having electrodes, through which the electronic module is attached to or detached from the sensing patch; and
conductive means, electrically coupled with the electrodes for making electrical contacts to skin.

16. The system of claim 15, wherein the electrodes of the electronic module are magnetized electrodes.

17. The system of claim 15, wherein the conductive means comprises conductive adhesive gel.

18. The system of claim 15, wherein the bio-signal capturing device further comprises holding means for holding the conductive means firmly to skin.

19. The system of claim 18, wherein the holding means comprises medical adhesive tape, elastic band or elastic string.

20. The system of claim 1, wherein the bio-signal capturing device comprises:
a sensing patch;
an electronic module; and
at least one socket connector, through which the electronic module is attached to or detached from the sensing patch.

21. The system of claim 1, wherein the event capturing device captures one or more events in the following: voice recording, sound recording, still imagery, video recording, body/muscle movement or posture, electromagnetic field (EMF) exposure, geographic location, orientation/gesture, finger tapping, altitude, temperature, humidity, and air pressure.

22. The system of claim 1, wherein the event capturing device is running continuously.

23. The system of claim 1, wherein the event capturing device is activated by one or more in the following: user, voice, sound, scene change, body/muscle movement or posture, EMF exposure change, geographic location change, orientation change, altitude change, temperature change, humidity change, air pressure change, computer software, and preset conditions.

24. The system of claim 1, wherein the event-based data analysis is a bio-signal and event correlation analysis based on the time reference of the captured bio-signal and the event marker.

25. The system of claim 1, wherein the event-based data analysis is multi-dimensional pattern recognition based on a priori characterization and modeling of the captured bio-signal and the event marker.

26. The system of claim 1, further comprising an analog-to-digital converter (ADC) utilized by the bio-signal capturing device and the event capturing device for converting input continuous physical quantity to a digital number that represents an amplitude of the quantity.

27. The system of claim 26, wherein at least one of the bio-signal capturing device and the event capturing device adopts dynamic gain switching during sampling of the ADC.

28. The system of claim 26, wherein at least one of the bio-signal capturing device and the event capturing device adopts non-uniform quantization for sampling of the ADC.

29. The system of claim 26, wherein at least one of the bio-signal capturing device and the event capturing device adopts data scrambling to prevent consecutive sampling data points being closest neighbors during communication; and the data recording device adopts spike removal within a corrupted data packet to reduce noise energy.

30. The system of claim 26, wherein at least one of the bio-signal capturing device and the event capturing device adopts collision detection, and adopts skip-forward algorithm upon collision detection.
